(19) United States
(12) Patent Application Publication
(10) Pub. No.: US 2010/0210675 A1
(43) Pub. Date: Aug. 19, 2010

(54) SOLVENT-FREE CRYSTALLINE FORM OF NALTREXONE

(75) Inventors: Ulrich Weigl, Hilzingen (DE); Ulf Költz, Tengen (DE)

Correspondence Address: HOFFMANN & BARON, LLP, 6900 JERICHO TURNPIKE, SYOSSET, NY, 11791, US

(73) Assignee: CILAG AG, Schaffhausen (CH)

(21) Appl. No.: 12/597,818

(22) PCT Filed: Apr. 27, 2007
(86) PCT No.: PCT/CH07/00203

§ 371 Date: Apr. 26, 2010

(51) Int. Cl. C07D 489/08; A61K 31/485; A61P 25/30

(52) U.S. Cl. 514/282; 546/45

(57) ABSTRACT

Solvent-free crystalline polymorphic form of naltrexone, characterized in that it has the XRD data listed in Table 1, and a method for the preparation of this polymorphic form; and a method for converting this polymorphic form of naltrexone into a known polymorphic form of naltrexone.

### CROSS-REFERENCE TO RELATED APPLICATIONS

This application is a National Phase Application of PCT/CH2007/000203 filed on Apr. 27, 2007, which is hereby incorporated by reference in its entirety herein.

### FIELD OF INVENTION

The present invention relates to a novel solvent-free crystalline polymorphic form of naltrexone and to processes for preparing this polymorphic form.

### BACKGROUND OF INVENTION

Naltrexone and the derivatives and salts thereof, for example naltrexone hydrochloride, N-methyl naltrexone bromide (methyl naltrexone) or naltrexone methobromide, are known pharmaceutically active compounds which are used in particular to reduce psychological dependency in drug abuse. The compound naltrexone as a free base corresponds to the chemical formula below:

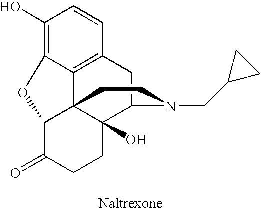

Numerous polymorphic forms of the free base of naltrexone, particularly solvates, and the preparation thereof are described in WO 2004/108084.

### SUMMARY OF INVENTION

It has now been found that a novel solvent-free crystalline polymorphic form of naltrexone can be obtained from ester compounds, in particular from (C₁-C₈) alkyl acetates, (C₁-C₈) alkyl butyrates and/or (C₁-C₈) alkyl benzoates, by crystallisation. This novel polymorphic form of naltrexone has some surprising and positive properties. The expression “solvent-free form” means that this form is neither a solvate nor a hydrate. The surprising and positive properties of this novel polymorphic form consist inter alia in the fact that it crystallises out of strongly coloured reaction solutions as a colourless product with very high HPLC purity and is highly stable.

Crystallisation generally proceeds in a simple manner and with high volume and reaction yields. In addition, dynamic vapour sorption demonstrates only very slight water take-up, even under highly hygroscopic conditions, which is of particular significance for the practical processing and use of the modification according to the invention, wherein any water that is taken up can be very easily eliminated again or removed by drying. The novel polymorphic form is therefore particularly suitable for formulations of naltrexone in which in accordance with the usual customer specifications the water content in the end product needs to be as low as possible.

### DETAILED DESCRIPTION

The present invention is defined in the claims. The present invention relates in particular to a novel solvent-free crystalline polymorphic form of naltrexone, which is characterised in that it has the following XRD data listed in Table 1:

TABLE 1

| 2 theta/deg | d/Ang. | I | I/I(max) in % |
|---|---|---|---|
| 7 | 12.63 | 83 | 10 |
| 7.6 | 11.63 | 280 | 32 |
| 8.4 | 10.53 | 270 | 31 |
| 10.6 | 8.35 | 870 | 100 |
| 12.6 | 7.03 | 700 | 80 |
| 13.6 | 6.51 | 320 | 37 |
| 14.9 | 5.95 | 430 | 49 |
| 16.3 | 5.44 | 710 | 82 |
| 17.3 | 5.13 | 420 | 48 |
| 17.7 | 5.01 | 840 | 97 |
| 18.8 | 4.72 | 230 | 26 |
| 19.5 | 4.55 | 310 | 36 |
| 21.4 | 4.15 | 400 | 46 |
| 22.3 | 3.99 | 540 | 62 |
| 23.2 | 3.83 | 250 | 29 |
| 23.6 | 3.77 | 210 | 24 |
| 25 | 3.56 | 190 | 22 |
| 25.6 | 3.48 | 190 | 22 |
| 26.5 | 3.36 | 290 | 33 |
| 29.3 | 3.05 | 230 | 26 |
| 33.0 | 2.71 | 100 | 11 |
| 36.3 | 2.47 | 130 | 15 |

Naltrexone of any purity can be used as the starting product. If the crude products are very impure (purity<80%) the crystallisation may need to be repeated.

For the crystallisation process the naltrexone starting product or the crude naltrexone product is dissolved in the solvent at elevated temperature, preferably at the reflux temperature of the solvent, in a concentration of preferably 1 (one) gram/100 grams to 50 grams/100 grams of solvent, wherein the solution is preferably stirred at the solution temperature for 10 minutes to 24 hours. It is then allowed to cool to room temperature, causing the polymorphic form according to the invention to crystallise out. To this end the solution is preferably cooled to a temperature in the range from room temperature, approximately 20° C., to −20° C.

In that respect the present invention relates to a process for preparing a solvent-free crystalline polymorphic form of naltrexone which is characterised in that as the starting product any naltrexone, preferably having a purity of at least 80% (purity≧80%), is dissolved in a solvent containing at least one ester compound or a mixture of ester compounds at elevated temperature, preferably at the reflux temperature of the solvent, preferably stirred at the solution temperature for ten minutes to 24 hours and then allowed to cool, wherein the polymorphic form according to the invention crystallises out.

The solvent preferably contains at least 80 wt. %, preferably at least 90 wt. %, of an ester compound or a mixture of ester compounds.

The naltrexone starting product is preferably dissolved in the solvent in a concentration of preferably 1 gram/100 grams to 50 grams/100 grams of solvent.

The mixture is preferably stirred at the solution temperature for 30 minutes to 12 hours and then allowed to cool to a temperature preferably in the range from approximately 20° C. to −20° C.

Ester compounds, in particular (C₁-C₈) alkyl acetates, preferably methyl acetate, ethyl acetate, propyl acetate, butyl acetate; (C₁-C₈) alkyl butyrates, preferably methyl butyrate, ethyl butyrate, propyl butyrate, butyl butyrate; (C₁-C₈) alkyl benzoates, preferably methyl benzoate, ethyl benzoate, propyl benzoate, butyl benzoate, are used as solvents for the crystallisation according to the invention. By preference methyl acetate, ethyl acetate, propyl acetate, butyl acetate; methyl butyrate, ethyl butyrate, methyl benzoate, ethyl benzoate or a mixture of these compounds is used, preferably methyl acetate, ethyl acetate, ethyl butyrate or a mixture of these compounds.

The invention further relates to a process for converting the polymorphic form according to the invention into a polymorphic form known per se, which is characterised in that the polymorphic form according to the invention is suspended in an alcohol, preferably a (C₁-C₄) alcohol, preferably methanol, ethanol, propanol, butanol, or in a ketone, preferably acetone, or in a mixture of these compounds, preferably in methanol, ethanol or acetone or in a mixture of these compounds, until the form according to the invention has converted into the known polymorph. Such a known form is described for example in US 2006/0142320, FIG. B. The suspension process causes a suspension to form.

The product is preferably suspended at slightly elevated temperature, preferably at a temperature in the range from −20° C. to +40° C., for a period of approximately 10 minutes to 24 hours, during which time the form known per se forms almost quantitatively. In order to isolate the product formed the suspension is preferably cooled to at least room temperature, preferably to a temperature in the range from room temperature (approximately 20° C.) to −20° C.

This process also offers the possibility of preparing a polymorph known per se by first preparing the polymorph according to the invention and then converting this into the polymorph known per se by suspension, for example in methanol.

This process offers the particular advantage of preparing the polymorph known per se in a very gentle manner and in a very pure form, without a partial decomposition of the naltrexone being observed, as is the case in the conventional recrystallisation of naltrexone owing to the use of elevated temperature.

The present invention also relates to the use of the polymorphs prepared according to the invention as therapeutic agents and the use of the polymorphs prepared according to the invention to produce a therapeutic agent suitable for pharmaceutical administration, in particular to reduce psychological dependency in drug abuse.

The following examples illustrate the invention without limiting its scope.

### Example 1

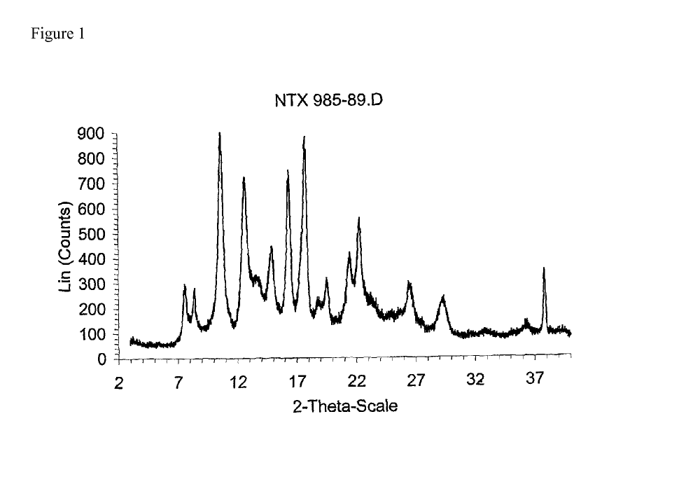

10 g of crude naltrexone are suspended in 50 g of ethyl acetate and refluxed. After refluxing for 1 hour (h) the solution obtained is cooled to 0° C. to 4° C. within 3 to 4 h and stirred for a further 1 to 2 h. The crystalline solid is siphoned off and dried under vacuum. 8 g of naltrexone are isolated. The XRD data for NTX (naltrexone) 985-89.D is shown in FIG. 1. The XRD data obtained corresponds to the values listed in Table 1.

### Example 2

2 g of crude naltrexone are suspended in 12 g of methyl acetate and refluxed until all naltrexone is dissolved. The solution obtained is cooled to 20-25° C. within 1 h. The crystalline solid is siphoned off and dried under vacuum. 1.0 g of naltrexone is isolated. The XRD data obtained corresponds in principle to the values listed in Table 1.

### Example 3

40 g of crude naltrexone are suspended in 136 g of methanol; then the mixture is stirred for 3 h at 15° C. The mixture is then stirred for a further 2 h at 0° C. The crystalline solid is siphoned off and dried under vacuum. 37.5 g of the naltrexone known per se, as described in US 2006/0142320, FIG. B, are isolated.

1. Solvent-free crystalline polymorphic form of naltrexone, characterised in that it has the XRD data listed in Table 1 below:
TABLE 1

| 2 theta/deg | D/Ang. | I | I/I (max) in % |
|---|---|---|---|
| 7 | 12.63 | 83 | 10 |
| 7.6 | 11.63 | 280 | 32 |
| 8.4 | 10.53 | 270 | 31 |
| 10.6 | 8.35 | 870 | 100 |
| 12.6 | 7.03 | 700 | 80 |
| 13.6 | 6.51 | 320 | 37 |
| 14.9 | 5.95 | 430 | 49 |
| 16.3 | 5.44 | 710 | 82 |
| 17.3 | 5.13 | 420 | 48 |
| 17.7 | 5.01 | 840 | 97 |
| 18.8 | 4.72 | 230 | 26 |
| 19.5 | 4.55 | 310 | 36 |
| 21.4 | 4.15 | 400 | 46 |
| 22.3 | 3.99 | 540 | 62 |
| 23.2 | 3.83 | 250 | 29 |
| 23.6 | 3.77 | 210 | 24 |
| 25 | 3.56 | 190 | 22 |
| 25.6 | 3.48 | 190 | 22 |
| 26.5 | 3.36 | 290 | 33 |
| 29.3 | 3.05 | 230 | 26 |
| 33.0 | 2.71 | 100 | 11 |
| 36.3 | 2.47 | 130 | 15 |

2. Process for preparing the polymorphic form of naltrexone according to claim 1, characterised in that as the starting product any naltrexone, preferably having a purity of at least 80% (purity≧80%), is dissolved in a solvent containing at least one ester compound or a mixture of ester compounds at elevated temperature, preferably at the reflux temperature of the solvent, preferably stirred at the solution temperature for ten minutes to 24 hours and then allowed to cool, wherein the polymorphic form of naltrexone according to claim 1 crystallises out.

3. Process according to claim 1, characterised in that the solvent contains at least 80 wt. %, preferably at least 90 wt. %, of an ester compound or a mixture of ester compounds.

4. Process according to claim 2, characterised in that the naltrexone starting product is dissolved in the solvent in a concentration of 1 gram/100 grams to 50 grams/100 grams of solvent.

5. Process according to claim 2, characterised in that the solution is stirred at the solution temperature for 30 minutes to 12 hours and then allowed to cool to a temperature in the range from approximately 20° C. to −20° C.

6. Process according to claim 2, characterised in that at least one ester compound, which is selected from the group containing (C₁-C₈) alkyl acetates, preferably methyl acetate, ethyl acetate, propyl acetate, butyl acetate; (C₁-C₈) alkyl butyrates, preferably methyl butyrate, ethyl butyrate, propyl butyrate, butyl butyrate; (C₁-C₈) alkyl benzoates, preferably methyl benzoate, ethyl benzoate, propyl benzoate, butyl benzoate, is used as the solvent.

7. Process according to claim 2, characterised in that methyl acetate, ethyl acetate, propyl acetate, butyl acetate; methyl butyrate, ethyl butyrate, methyl benzoate, ethyl benzoate or a mixture of these compounds is used as the solvent, preferably methyl acetate, ethyl acetate, ethyl butyrate or a mixture of these compounds.

8. Process for converting the polymorphic form of naltrexone according to claim 1 into a polymorphic form known per se, characterised in that the polymorphic form of naltrexone according to claim 1 is suspended in an alcohol, preferably a (C₁-C₄) alcohol, preferably methanol, ethanol, propanol, butanol, or in a ketone, preferably acetone, or in a mixture of these compounds, preferably in methanol, ethanol or acetone or in a mixture of these compounds, until the form according to the invention has converted into the known polymorph.

9. Process according to claim 8, characterised in that the product is suspended at slightly elevated temperature, preferably at a temperature in the range from −20° C. to +40° C., for a period of approximately 10 minutes to 24 hours.

10. Process according to claim 8, characterised in that in order to isolate the product formed the suspension is cooled to at least room temperature, preferably to a temperature in the range from room temperature to −20° C.

11. Use of the polymorphic form of naltrexone according to claim 1 as a therapeutic agent, in particular to reduce psychological dependency in drug abuse.

12. Use of the polymorphic form of naltrexone according to claim 1 to produce a therapeutic agent suitable for pharmaceutical administration which is particularly effective in reducing psychological dependency in drug abuse.
